(19) United States
(12) Patent Application Publication
(10) Pub. No.: US 2003/0187028 A1
(43) Pub. Date: Oct. 2, 2003

(54) Medicament for viral diseases

(76) Inventors: Michael Brands, Wuppertal (DE); Susanne Nikolic, Monheim (DE); Peter Eckenberg, Wuppertal (DE); Marcus Bauser, Wuppertal (DE); Johannes Kaulen, Odenthal (DE); Arnold Paessens, Haan (DE); Erwin Graef, Wulfrath (DE); Olaf Weber, Woodbridge, CT (US); Stefan Lottmann, Wuppertal (DE); Karl-Heinz Schlemmer, Wuppertal (DE)

Correspondence Address: JEFFREY M. GREENMAN, VICE PRESIDENT, PATENTS AND LICENSING, BAYER CORPORATION, 400 MORGAN LANE, WEST HAVEN, CT, 06516, US

(21) Appl. No.: 10/220,110

(22) PCT Filed: Feb. 19, 2001
(86) PCT No.: PCT/EP01/01825

(30) Foreign Application Priority Data
Feb. 28, 2000 (DE) 100 09 408.2
Jul. 6, 2000 (DE) 100 32 874.1

(51) Int. Cl. A61K 31/4439; A61K 31/42; C07D 413/02

(52) U.S. Cl. 514/340; 514/378; 548/235; 546/272.1

(57) ABSTRACT

Isoxazoles are highly effective anti-viral agents. Combinations of isoxazoles, dihydropyrimidines and/or lamivudine and, optionally, interferon inhibit the proliferation of HBV viruses better than conventional agents.

The present invention relates to novel isoxazoles and to combinations of A) non-nucleosidic inhibitors from the class of isoxazoles, B) other antiviral active substances such as (i) dihydropyrimidines and/or (ii) nucleoside analogs such as, for example, lamivudine, and, where appropriate, C) immunomodulators such as, for example, interferon (the combinations are double, triple or quadruple combinations), to a process for preparing the isoxazoles and combinations and to their use as medicaments, in particular for the treatment and prophylaxis of HBV infections.

“Combinations” mean for the purpose of the invention not only dosage forms which contain all the components (so-called fixed combinations), and combination packs which contain the components separate from one another, but also components which are administered simultaneously or sequentially, as long as they are employed for the treatment or prophylaxis of the same disease.

The hepatitis B virus belongs to the family of hepadna viruses. It causes an acute and/or a persistent/progressive chronic disease. Many other clinical manifestations in the pathological state are also caused by the hepatitis B virus—in particular chronic inflammation of the liver, cirrhosis of the liver and hepatocellular carcinoma. In addition, coinfection with the hepatitis delta virus may have adverse effects on the progress of the disease.

Several possibilities have already been proposed for virus inhibition:

1. inhibition of the virus by dihydropyrimidines which bring about a large reduction in the viral DNA and in the viral core protein;

2. inhibition of HBV polymerase by analogs of the substrates of this enzyme such as lamivudine, FTC, adefovir dipivoxil, abacavir, β-L-FDDC, L-FMAU and BMS 200 475;

3. inhibition of HBV by immunological principles such as, for example, the treatment of chronic hepatitis by interferon;

4. inhibition by other active substances whose modes of action are not known or are the subject of speculation, such as, for example, AT-61=N-[(1E)-2-chloro-2-phenyl-1-(1-piperidinylcarbonyl)ethenyl]benzamide, which evidently intervenes in the process of packaging the pregenomic RNA into the incomplete core particles; cf. King et al., Antimicrob. Agents and Chemother. 42, 3179-3186 (1998);

5. stimulation of the host's immune defenses, such as, for example, with thymosin-α.

The only agents approved for the treatment of chronic hepatitis are interferon and lamivudine. However, interferon has only moderate activity and has unwanted side effects; although lamivudine has good activity, resistance develops rapidly during treatment and a rebound effect occurs in most cases after discontinuation of the therapy. Combinations of interferon with lamivudine have no synergistic activity.

Therapeutic agents employed to date for the treatment of HBV-infected patients, such as, for example, interferon or lamivudine, are employed as monotherapy. It is known from clinical studies that combinations of the two inhibitors have no advantage for controlling HBV diseases.

No generally available clinical experience is available as yet for adefovir dipivoxil, BMS 200 475 and the other inhibitors mentioned above.

Novel agents for better and effective therapy are therefore desirable.

WO 99/45908 has disclosed isoxazoles unsubstituted in the 3 position, e.g. leflunomide (═N-(4-trifluoromethylphenyl)-5-methylisoxazole-4-carboxamide), with an antiviral effect, inter alia against hepatitis viruses. However, our investigations with these compounds have not revealed any activity against the hepatitis B virus.

As described in WO 99/45908, leflunomide is rapidly metabolized in vivo by ring opening to N-(4-trifluoromethylphenyl)-2-cyano-4-oxobutryamide. This isomerization is possible only for isoxazoles which have a hydrogen atom on the carbon atom adjacent to the nitrogen atom of the isoxazole ring (3 position).

It has been found, surprisingly, that isoxazoles substituted in the 3 position are distinctly superior to the isoxazoles of WO 99/45908 and are highly effective against hepatitis viruses. It has additionally been found that combinations of A) isoxazoles, B) other HBV-antiviral active substances and, where appropriate, C) immunomodulators have the prior art disadvantages only partly or not at all.

The invention thus relates to compounds of the formula

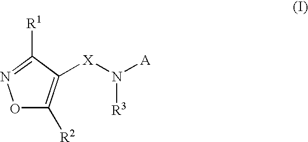

in which

R¹ and R² are, independently of one another, alkyl which is optionally substituted by one or more halogen atoms,

X is a divalent radical from the series consisting of C═Y, —N(R⁴)—C(═Y)—, —CH₂— or a group of the formula —(CH₂)ₙC(═Y)—,

n is an integer from 1 to 4,

R³ and R⁴ are, independently of one another, hydrogen or optionally halogen-substituted alkyl,

Y is an oxygen or sulfur atom and

A is aryl or 6-membered hetaryl which is optionally substituted by 1 to 3 radicals which are selected, independently of one another, from the series halogen, alkyl, alkoxy, alkylthio, alkoxycarbonyl, aminocarbonylamino, mono- and dialkylamino, cyano, amino, mono- and dialkylaminocarbonyl—in the case of substitution in the o position from the series halogen, alkyl, alkoxy, alkylthio.

The following applies to the compounds I:

Alkyl and the alkyl moieties in mono- and dialkylamino and in mono- and dialkylaminocarbonyl are within the framework of the invention a linear or branched alkyl radical having 1 to 8, preferably 1 to 6, carbon atoms, such as, for example, methyl, ethyl, propyl, isopropyl, tert-butyl, n-pentyl, n-hexyl, 2-ethylhexyl or n-octyl.

Alkoxy is within the framework of the invention a linear or branched alkoxy radical having 1 to 6, preferably 1 to 4, carbon atoms, such as, for example, methoxy, ethoxy, propoxy, isopropoxy, tert-butoxy, n-pentoxy and n-hexoxy.

Alkylthio is within the framework of the invention a linear or branched alkylthio radical having 1 to 6, preferably 1 to 4, carbon atoms, such as, for example, methylthio, ethylthio and propylthio.

Alkoxycarbonyl is within the framework of the invention a linear or branched alkoxycarbonyl radical having 1 to 6, preferably 1 to 4, carbon atoms, such as, for example, methoxycarbonyl, ethoxycarbonyl, propoxycarbonyl, isopropoxycarbonyl, tert-butoxycarbonyl, n-pentoxycarbonyl and n-hexoxycarbonyl.

Halogen is within the framework of the invention fluorine, chlorine, bromine or iodine.

The preferred halogenated alkyl is trifluoromethyl.

Aryl is generally an aromatic radical having 6 to 10 carbon atoms, preferably phenyl and naphthyl.

Hetaryl is within the framework of the invention preferably pyridyl or pyrimidyl.

Preferred compounds of the formula (I) are those in which

R¹ and R² are, independently of one another, optionally halogen-substituted C₁-C₈-alkyl,

X is a divalent radical from the series C═Y and CH₂,

R³ and R⁴ are, independently of one another, hydrogen or optionally halogen-substituted C₁-C₆-alkyl,

Y is an oxygen or sulfur atom and

A is phenyl, pyridyl or pyrimidyl, which are optionally substituted by 1 to 3 radicals from the series halogen, C₁-C₆-alkyl, C₁-C₆-alkoxy, C₁-C₆-alkylthio, C₁-C₆-alkoxycarbonyl, carbamoyl, mono-C₁-C₆-alkylaminocarbonyl, di-C₁-C₆-alkylaminocarbonyl, cyano—in the case of substitution in the o position from the series halogen, C₁-C₆-alkyl, C₁-C₆-alkoxy, C₁-C₆-alkylthio.

Particularly preferred compounds of the formula (I) are those in which

R¹ and R² are, independently of one another, C₁-C₆-alkyl or trifluoromethyl,

X is C═Y,

R³ and R⁴ are, independently of one another, hydrogen or C₁-C₆-alkyl, preferably hydrogen or methyl,

Y is an oxygen or sulfur atom and

A is mono- to trisubstituted, preferably 3,4- or 3,5-disubstituted, phenyl or pyridyl, the substituents of which are selected independently of one another from the series alkyl, halogen, CF₃, in particular 3-methyl-4-fluoro- and 3-chloro-4-fluorophenyl.

The isoxazoles of the invention can be prepared from the corresponding acid chlorides 2 by reaction with an amine HNAR³

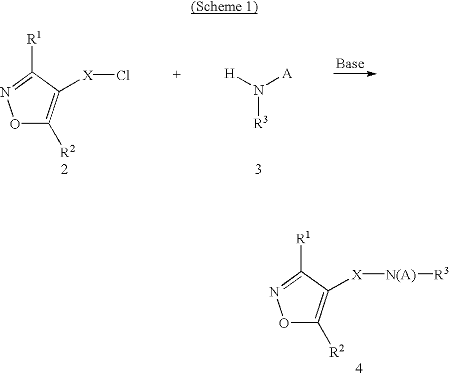

The heterocyclic building block 2 can be synthesized for example in analogy to G. Storck, J. E. McMurry, J. Am. Chem. Soc. 1967, 89, 5461 as shown in the following scheme:

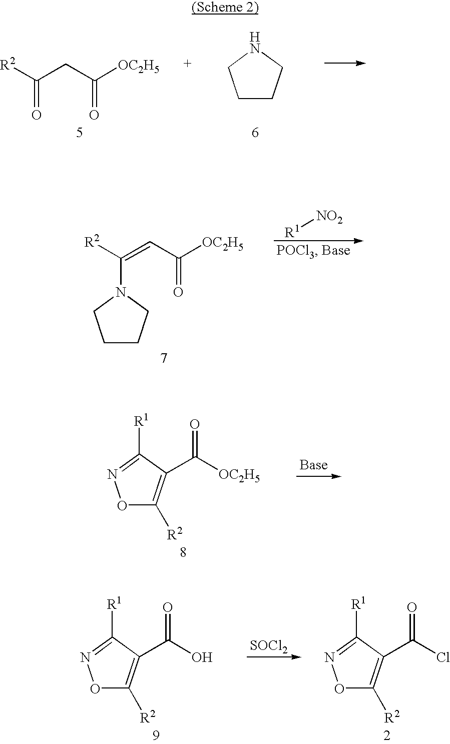

For this purpose, for example, the keto ester 5 is converted with pyrroldine 6, under water-abstracting conditions into the enamino ester 7 which reacts with an aliphatic nitro compound in the presence of base such as, for example, triethylamine and a water-abstracting agent such as phenyl isocyanate or phosphorus oxychloride to give the isoxazole 8. The ethyl ester can then be cleaved for example with aqueous sodium hydroxide solution, and the resulting acid 9 can be converted for example by treatment with thionyl chloride into the acid chloride.

3-(3,5-dimethyl-4-isoxazolyl)acetyl chloride and -propanoyl chloride can be prepared for example in analogy to the literature (J. Org. Chem. 59, 2882-2884 (1994), Ann. Chim. 26 (7), 340 (1902)).

Anilines or heterocyclic amines which can be purchased can be used as amine component 3.

The bases which can generally be employed for the reactions in schemes 1 and 2 are sodium or lithium bistrimethylsilylamide; alkali metal hydroxides such as sodium hydroxide, lithium hydroxide or potassium hydroxide; sodium bicarbonate; sodium hydride; organic tri-(C₁-C₆)alkylamines such as trimethylamine or diisopropylethylamine; heterocycles such as 1,4-diazabicyclo[5.4.0]undec-7-ene (DBU), pyridine, diaminopyridine, methylpiperidine or N-methylmorpholine.

Preferred bases for the reactions in scheme 1 comprise organic amines such as triethylamine, diisopropylethylamine or N-methylmorpholine, which may also be carrier-bound, such as, for example, morpholinomethyl-polystyrene.

Preferred bases for the reactions in scheme 2 comprise lithium hydroxide, pyridine, diisopropylethylamine and triethylamine.

The thioamides (formula I with Y═S) can be synthesized by treating the amides 4 with Lawesson's reagent (=2,4-bis-(4-methoxyphenyl)-1,3,2,4 dithiaphosphetane 2,4 disulfide; cf. R. Shabana et al., Tetrahedron 1980 (36), 3047-3051); the reaction can take place in toluene at elevated temperature.

The ureas [X═—N(R⁴)—C(═Y)—] can be synthesized employing, for example, 3-amino-2,5-dimethylisoxazole as starting material (A. Pascual, Helv. Chim. Acta 1989 (72), 556-569) which, after conversion to the carbamoyl chloride, is reacted with amines HNAR³ in an analogous manner.

The amines (X═CH₂) can be obtained from the corresponding carboxamides described in scheme 1 by reduction for example with borane/dimethylsulfide complex (J. March, Advanced Organic Chemistry, 4th edition, New York 1992, p. 1212).

The reactions in schemes 1 and 2 can be carried out in inert organic solvents. These comprise saturated linear, branched and cyclic hydrocarbons such as hexane, cyclohexane or petroleum fractions, alcohols such as methanol, ethanol or isopropanol, ethers such as diethyl ether, 1,4-dioxane or tetrahydrofuran, halogenated hydrocarbons such as dichloromethane, chloroform, tetrachloromethane, 1,2-dichloroethane, trichloroethane or tetrachloroethane, aromatic hydrocarbons such as benzene, toluene or xylene, dipolar aprotic solvents such as nitromethane, dimethylformamide or acetonitrile, or mixtures thereof. Dichloromethane, chloroform, 1,2-dichloroethane, toluene, ethanol and dimethylformamide are particularly preferred.

Preferred solvents for the reactions in scheme 1 comprise chlorinated hydrocarbons such as dichloromethane, chloroform, 1,2-dichloromethane and ethers such as tetrahydrofuran. The reactions in scheme 2 are preferably carried out in aromatic hydrocarbons such as toluene, chlorinated hydrocarbons such as dichloromethane, chloroform, 1,2-dichloroethane, ethers such as tetrahydrofuran or alkanols such as ethanol.

The reactions in schemes 1 and 2 are generally carried out in a temperature range from 0 to 150, preferably from 0 to 90° C. The reactions can be carried out under atmospheric, reduced or elevated pressure (e.g. 0.5 to 5 bar); atmospheric pressure is generally used.

The invention thus relates further to a process for preparing compounds of the formula (I) in which

R¹ to R³ and A have the meanings indicated above,

X is C═Y or or a group of the formula —(CH₂)ₙC(═Y)—,

Y is an oxygen atom and

N is an integer from 1 to 4,

by reacting acid chlorides of the formula

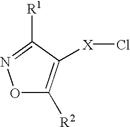

in which

R¹ and R², and X and Y, have the meanings indicated above,

with amines of the formula NHAR³,

in which A and R³ have the meanings indicated above.

The invention further relates to a process for preparing compounds of the formula (I) in which

R¹ to R³ and A have the meanings indicated above, and

X is C═Y and

Y is a sulfur atom,

by treating compounds of the formula (I) in which

R¹ to R³ and A have the meanings indicated above,

X is C═Y and

Y is an oxygen atom,

with Lawesson's reagent.

The invention further relates to a process for preparing compounds of the formula (I) in which

R¹ to R³ and A have the meanings indicated above, and

X is CH₂,

by reducing compounds of the formula (I) in which

R¹ to R³ and A have the meanings indicated above, and

X is C═Y and

Y is an oxygen atom.

The invention further relates to a process for preparing compounds of the formula (I) in which

R¹ and R⁴ and A have the meanings indicated above, and

X is —N(R⁴)—C(═Y)— and

Y is an oxygen atom,

by reacting compounds of the formula

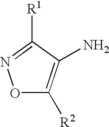

in which

R¹ and R² have the meanings indicated above,

with carbonyl group donors and amines of the formula NHAR³,

in which A and R³ have the meanings indicated above.

The term “carbonyl group donors” comprises for the purpose of the invention for example trichloromethyl chloroformate, carbonyldiimidazole and phosgene.

The invention further relates to a process for preparing compounds of the formula (I) in which

R¹ to R⁴ and A have the meanings indicated above, and

X is —N(R⁴)—C(═Y)— and

Y is a sulfur atom,

by reacting compounds of the formula

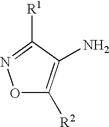

in which

R¹ and R² have the meanings indicated above,

with thiocarbonyl group donors and amines of the formula NHAR³,

in which A and R³ have the meanings indicated above.

The term “thiocarbonyl group donors” comprises for the purpose of the invention, for example, N,N′-thiocarbonyldiimidazole and thiophosgene.

The invention further relates to combinations of A) at least one isoxazole, B) at least one HBV-antiviral active substance different from A, preferably (i) an HBV DNA inhibitor or HBV core protein inhibitor and/or (ii) an HBV polymerase inhibitor, and optionally (C) at least one immunomodulator. The invention thus relates to combinations of nucleosidic and non-nucleosidic inhibitors and, where appropriate, immunomodulators for the treatment and prophylaxis of HBV infections, and to the use of these combinations for the treatment of HBV-induced diseases.

Suitable isoxazoles A are the compounds I described above.

HBV DNA inhibitors or HBV core protein inhibitors B (i) are those non-nucleosidic inhibitors which show intra- and extracellular inhibition of HBV DNA and at least of the half-life of the HBV core protein in the cell.

Preferred HBV core protein inhibitors B (i) are, for example, dihydropyrimidines, preferably those described in German published specifications 198 17 264 (=WO 99/54 326), 198 17 265 (=WO 99/54 312) and 198 17 262 (=WO 99/54 329).

Preferred dihydropyrimidines B (i) correspond, for example, to the formula

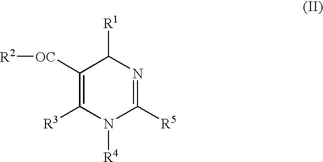

or the isomeric form thereof

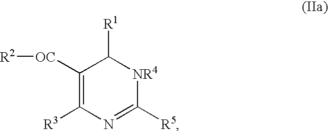

and the salts thereof, in which

R¹ is phenyl, furyl, thienyl, triazolyl, pyridyl, cycloalkyl having 3 to 6 carbon atoms or radicals of the formulae

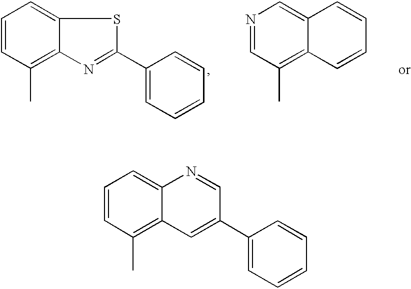

where the ring systems mentioned above are optionally substituted one or more times, identically or differently, by substituents selected from the group of halogen, trifluoromethyl, nitro, cyano, trifluoromethoxy, carboxyl, hydroxyl, C₁-C₆-alkoxy, C₁-C₆-alkoxycarbonyl and C₁-C₆-alkyl, where the alkyl radical in turn may be substituted by aryl having 6 to 10 carbon atoms or halogen, and the mentioned ring systems are optionally substituted by —S—R⁶, —NR⁷R⁸, —CO—NR⁹R¹⁰, —SO₂—CF₃ and —A—CH₂—R¹¹,

in which

R⁶ is optionally halogen-substituted phenyl,

R⁷ to R¹⁰ are, independently of one another, hydrogen, phenyl, hydroxy-substituted phenyl, hydroxyl, C₁-C₆-acyl or C₁-C₆-alkyl, where the alkyl radical in turn may be substituted by hydroxyl, C₁-C₆-alkoxycarbonyl, phenyl or hydroxy-substituted phenyl,

A is a radical —O—, —S—, —SO— or —SO₂—,

R¹¹ is phenyl which is optionally substituted one or more times, identically or differently, by substituents selected from the group of halogen, nitro, trifluoromethyl, C₁-C₆-alkyl and C₁-C₆-alkoxy,

R² denotes a radical of the formulae —XR¹² or —NR¹³R¹⁴,

in which

X is a single bond or oxygen,

R¹² is hydrogen, straight-chain or branched C₁-C₆-alkoxycarbonyl, a straight-chain, branched or cyclic, saturated or unsaturated C₁-C₈-hydrocarbon radical which optionally contains one or two identical or different hetero chain members from the group of —O—, —CO—, —NH—, —N—(C₁-C₄-alkyl)-, —S— or —SO₂— and which is optionally substituted by halogen, nitro, cyano, hydroxyl, aryl having 6 to 10 carbon atoms, aralkyl having 6 to 10 carbon atoms, heteroaryl or a group of the formula —NR¹⁵R¹⁶,

in which

R¹⁵ and R¹⁶ are, independently of one another, hydrogen, benzyl or C₁-C₆-alkyl,

R¹³ and

R¹⁴ are, independently of one another, hydrogen, C₁-C₆-alkyl or cycloalkyl having 3 to 6 carbon atoms,

R³ is hydrogen, amino or a radical of the formula

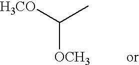

formyl, cyano, hydroxy-substituted C₁-C₆-alkylthio, trifluoromethyl or pyridyl or a straight-chain, branched or cyclic, saturated or unsaturated hydrocarbon radical having up to 8 carbon atoms which is optionally substituted one or more times, identically or differently, by aryloxy having 6 to 10 carbon atoms, azido, halogen, cyano, hydroxyl, carboxyl, C₁-C₆-alkoxycarbonyl, a 5- to 7-membered heterocyclic ring, C₁-C₆-alkylthio or C₁-C₆-alkoxy (where the alkylthio or alkoxy radical may in turn be substituted by azido, amino, hydroxyl) and/or by the group —(CO)ₐ—NR¹⁷R¹⁸,

in which

a is zero or 1,

R¹⁷ and R¹⁸ are, independently of one another, hydrogen or aryl having 6 to 10 carbon atoms, aralkyl having 6 to 10 carbon atoms or C₁-C₆-alkyl, each of which is optionally substituted by C₁-C₆-alkoxycarbonyl, amino, hydroxyl, phenyl or benzyl, where phenyl and benzyl are optionally substituted one or more times, identically or differently, by hydroxyl, carboxyl, C₁-C₆-alkyl or C₁-C₆-alkoxy, and/or C₁-C₆-alkyl is optionally substituted by —NH—CO—CH₃ or —NH—CO—CF₃,

or

R¹⁷ and R¹⁸ together with the nitrogen atom on which they are located are a morpholinyl, piperidinyl or pyrrolidinyl ring,

or

R³ is optionally methoxy-substituted phenyl

or

R² and R³ together are a radical of the formula

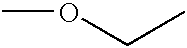

R⁴ is hydrogen, C₁-C₄-alkyl, C₂-C₄-alkenyl, benzoyl or acyl having 2 to 6 carbon atoms, preferably hydrogen, methyl, benzoyl or C₂-C₆-acyl, and

R⁵ is pyridyl, pyrimidyl or pyrazinyl, each of which may be substituted up to 3 times, identically or differently, by halogen, hydroxyl, cyano, trifluoromethyl, C₁-C₆-alkoxy, C₁-C₆-alkyl, C₁-C₆-alkylthio, carbalkoxy, C₁-C₆-acyloxy, amino, nitro, mono- or di-C₁-C₆-alkylamino.

Very particularly preferred dihydropyrimidines B are the following compounds:

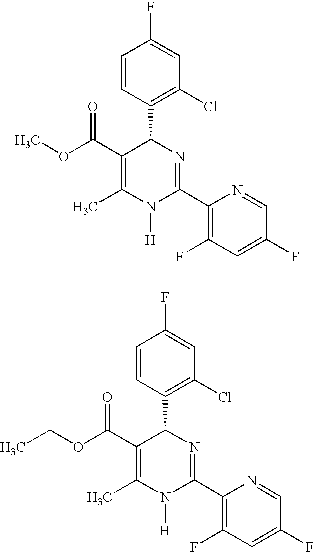

their isomeric forms and their salts.

The compounds II and IIa include the isomers of the formulae (II) and (IIa) and mixtures thereof. If R⁴ is hydrogen, the isomers (II) and (IIa) are present in tautomeric equilibrium:

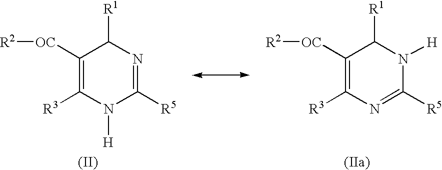

The above dihydropyrimidines II and IIa and various processes for their preparation are disclosed in German published specifications 198 17 264 (=WO 99/54 326) and 198 17 265 (=WO 99/54 312).

Further preferred dihydropyrimidines B (i) correspond to the formula

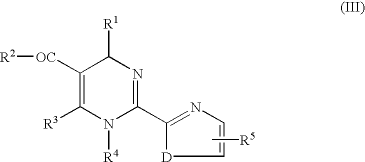

and the isomeric form thereof

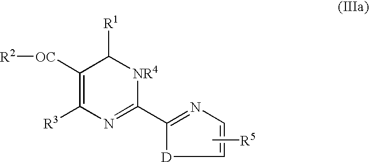

and/or the salts thereof, in which

R¹ is phenyl, furyl, thienyl, pyridyl, cycloalkyl having 3 to 6 carbon atoms or a radical of the formulae

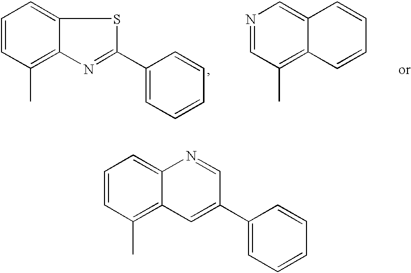

where the ring systems mentioned above are optionally substituted one or more times, identically or differently, by substituents selected from the group of halogen, trifluoromethyl, nitro, cyano, trifluoromethoxy, carboxyl, hydroxyl, C₁-C₆-alkoxy, C₁-C₆-alkoxycarbonyl and C₁-C₆-alkyl, where the alkyl radical in turn may be substituted by aryl having 6 to 10 carbon atoms or halogen, and/or the mentioned ring systems are optionally substituted by groups of the formulae —S—R⁶, —NR⁷R⁸, —CO—NR⁹R¹⁰, —SO₂—CF₃ and —A—CH₂—R¹¹,

in which

R⁶ is optionally halogen-substituted phenyl,

R⁷ to R¹⁰ are, independently of one another, hydrogen, phenyl, hydroxy-substituted phenyl, hydroxyl, C₁-C₆-acyl or C₁-C₆-alkyl, where the alkyl radical in turn may be substituted by hydroxyl, C₁-C₆-alkoxycarbonyl, phenyl or hydroxy-substituted phenyl,

A is a radical —O—, —S—, —SO— or —SO₂—,

R¹¹ is phenyl which is optionally substituted one or more times, identically or differently, by substituents selected from the group of halogen, nitro, trifluoromethyl, C₁-C₆-alkyl and C₁-C₆-alkoxy,

R² denotes a radical of the formulae —OR¹² or —NR¹³R¹⁴,

in which

R¹² is hydrogen, C₁-C₆-alkoxycarbonyl or a straight-chain, branched or cyclic, saturated or unsaturated C₁-C₈-hydrocarbon radical which optionally contains one or two identical or different hetero chain members from the group of —O—, —CO—, —NH—, —N—(C₁-C₄-alkyl)-, —S— and —SO₂— and which is optionally substituted by halogen, nitro, cyano, hydroxyl, aryl having 6 to 10 carbon atoms or aralkyl having 6 to 10 carbon atoms, heteroaryl or a group of the formula —NR¹⁵R¹⁶,

in which

R¹⁵ and R¹⁶ are, independently of one another, hydrogen, benzyl or C₁-C₆-alkyl,

R¹³ and R¹⁴ are, independently of one another, hydrogen, C₁-C₆-alkyl or cycloalkyl having 3 to 6 carbon atoms,

R³ is hydrogen, amino or a radical of the formula

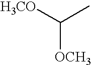

or formyl, cyano, hydroxy-substituted C₁-C₄-alkylthio, trifluoromethyl or a straight-chain, branched or cyclic, saturated or unsaturated hydrocarbon radical having up to 8 carbon atoms, which is optionally substituted one or more times, identically or differently, by aryloxy having 6 to 10 carbon atoms, azido, cyano, hydroxyl, carboxyl, C₁-C₆-alkoxycarbonyl, a 5- to 7-membered heterocyclic ring, C₁-C₆-alkylthio or C₁-C₆-alkoxy (where the alkylthio or alkoxy radical in turn can be substituted by azido, amino or hydroxyl) and/or by the group —(CO)ₐ—NR¹⁷R¹⁸,

in which

a is zero or 1,

R¹⁷ and R¹⁸ are, independently of one another, hydrogen or aryl, aralkyl having 6 to 10 carbon atoms or C₁-C₆-alkyl, which are optionally substituted by C₁-C₆-alkoxycarbonyl, amino, hydroxyl, phenyl or benzyl, where phenyl and benzyl are optionally substituted one or more times, identically or differently, by hydroxyl, carboxyl, C₁-C₆-alkyl or C₁-C₆-alkoxy, and/or C₁-C₆-alkyl is optionally substituted by —NH—CO—CH₃ or —NH—CO—CF₃,

or

R¹⁷ and R¹⁸ together with the nitrogen atom on which they are located are a morpholinyl, piperidinyl or pyrrolidinyl ring,

D is an oyxgen or sulfur atom and

R⁵ is hydrogen, halogen or straight-chain or branched alkyl having up to 6 carbon atoms.

The compounds III and IIIa may exist in stereoisomeric forms which either are related as image and mirror image (enantiomers) or are not related as image and mirror image (diastereomers). The compounds III and IIIa thus encompass both the enantiomers and the diastereomers, and the respective mixtures thereof. The racemic forms can, just like the diastereomers, be separated into the stereoisomerically homogeneous components in a known manner.

The dihydropyrimidines III and IIIa which contain in position 2 an optionally substituted oxazolyl or thiazolyl radical, and various processes for their preparation are disclosed in German published specification 198 17 262 (=WO 99/54 329).

The following applies to the compounds II, IIa, III and IIIa:

Alkyl per se and the alkyl moieties in mono- and dialkylamino and in mono- and dialkylaminocarbonyl are within the framework of the invention a linear or branched alkyl radical having 1 to 8, preferably 1 to 6, carbon atoms, such as, for example, methyl, ethyl, propyl, isopropyl, tert-butyl, n-pentyl, n-hexyl, 2-ethylhexyl or n-octyl.

Alkenyl is within the framework of the invention a straight-chain or branched alkenyl radical having 2 to 6, preferably 3 to 5, carbon atoms, such as, for example, ethenyl, propenyl, isopropenyl, tert-butenyl, n-pentenyl and n-hexenyl.

Cycloalkyl having 3 to 6 carbon atoms is within the framework of the invention cyclopropyl, cyclopentyl, cyclobutyl, cyclohexyl, preferably cyclopentyl and cyclohexyl.

Acyl is within the framework of the invention a straight-chain or branched acyl radical having 1 to 6, preferably 1 to 4, carbon atoms such as, for example, acetyl and propionyl.

Alkoxy is within the framework of the invention a linear or branched alkoxyl radical having 1 to 6, preferably 1 to 4, carbon atoms such as, for example, methoxy, ethoxy, propoxy, isopropoxy, tert-butoxy, n-pentoxy and n-hexoxy.

Alkylthio is within the framework of the invention a linear or branched alkylthio radical having 1 to 6, preferably 1 to 4, carbon atoms such as, for example, methylthio, ethylthio and propylthio.

Alkoxycarbonyl is within the framework of the invention a linear or branched alkoxylcarbonyl radical having 1 to 6, preferably 1 to 4, carbon atoms such as, for example, methoxycarbonyl, ethoxycarbonyl, propoxycarbonyl, isopropoxycarbonyl, tert-butoxycarbonyl, n-pentoxycarbonyl and n-hexoxycarbonyl.

Aralkyl is within the framework of the invention aralkyl having, preferably, 6 to 10, in particular 6, carbon atoms in the aryl moiety (preferably phenyl or naphthyl, in particular phenyl) and preferably 1 to 4, in particular 1 or 2, carbon atoms in the alkyl moiety, where the alkyl moiety can be linear or branched. Preferred aralkyl radicals are benzyl and phenethyl.

Aryl is within the framework of the invention an aromatic radical having 6 to 10 carbon atoms, preferably phenyl and naphthyl.

Heteroaryl is within the framework of the invention 5- to 7-membered rings with, preferably, 1 to 3, in particular 1 or 2, identical or different heteroatoms from the series oxygen, sulfur and nitrogen. Preferred examples comprise furyl, thiophenyl, pyrazolyl, imidazolyl, 1.2.3- and 1.2.4-triazolyl, oxazolyl, isoxazolyl, thiazolyl, isothiazolyl, 1.2.3-, 1.3.4-, 1.2.4- and 1.2.5-oxadiazolyl, pyrrolyl, pyridyl, pyrimidinyl, pyrazinyl, 1.3.5-, 1.2.4- and 1.2.3-triazinyl, 1.2.4-, 1.3.2-, 1.3.6- and 1.2.6-oxazinyl, in particular pyridyl and pyrimidyl.

Halogen is within the framework of the invention fluorine, chlorine, bromine or iodine.

The preferred halogenated alkyl is trifluoromethyl.

The compounds II or IIIa and III or IIIa may also be in the form of salts. Physiologically acceptable salts are preferred for the purposes of the invention.

Physiologically acceptable salts may be salts of the compounds II or IIa and III or IIIa with inorganic or organic acids. Preference is given to salts of inorganic acids such as, for example, hydrochloric acid, hydrobromic acid, phosphoric acid or sulfuric acid, or salts of organic carboxylic or sulfonic acids such as, for example, acetic acid, maleic acid, fumaric acid, malic acid, citric acid, tartaric acid, lactic acid, benzoic acid, or methanesulfonic acid, ethanesulfonic acid, phenylsulfonic acid, toluenesulfonic acid or napthalenedisulfonic acid.

Physiologically acceptable salts may likewise be metal or ammonium salts of the compounds II or IIa and III or IIIa. Particularly preferred examples are sodium, potassium, magnesium or calcium salts, and ammonium salts derived from ammonia or organic amines such as, for example, ethylamine, di- or triethylamine, di- or triethanolamine, dicyclohexylamine, dimethylaminoethanol, arginine, lysine, ethylenediamine or 2-phenylethylamine.

The invention further relates to combinations of

A) at least one isoxazole,

B) at least (i) one dihydropyrimidine and/or (ii) one HBV polymerase inhibitor and, where appropriate,

C) at least one immunomodulator.

The substances referred to as HBV polymerase inhibitors B (ii) for the purposes of the invention are those which, in the endogenous polymerase assay (Ph. A. Furman et al. in Antimicrobial Agents and Chemotherapy, Vol. 36 (No. 12), 2688 (1992)) lead to an inhibition of the formation of an HBV DNA double strand, so as to result in a maximum of 50% of the activity of the zero value:

Preferred HBV polymerase inhibitors B (ii) comprise, for example,

3TC=lamivudine=4-amino-1-[(2R-cis)-2-(hydroxymethyl)-1.3-oxathiolan-5-yl]-pyrimidin-2(1H)-one, compare European Patent 382 526 (=U.S. Pat. No. 5,047,407) and WO 91/11186 (=U.S. Pat. No. 5,204,466);

adefovir dipivoxil=9-{2-[[bis[(pivaloyloxy)-methoxy]-phosphinyl]-methoxy]-ethyl}-adenine, compare European Patent 481 214 (=U.S. Pat. Nos. 5,663,159 and 5,792,756), U.S. Pat. Nos. 4,724,233 and 4,808,716;

BMS 200 475=[1S-(1.α,3.α,4.β)]-2-amino-1.9-dihydro-9-[4-hydroxy-3-(hydroxymethyl)-2-methylene-cyclopentyl]-6H-purin-6-one, compare European Patent 481 754 (=U.S. Pat. Nos. 5,206,244 and 5,340,816), WO 98/09964 and 99/41275;

abacavir=(−)-(1S-cis)-4-[2-amino-6-(cyclopropylamino)-9H-purin-9-yl]-2-cyclopentene-1-methanol, compare European Patent 349 242 (=U.S. Pat. No. 5,049,671) and European Patent 434 450 (=U.S. Pat. Specification No. 5,034,394);

FTC=(2R-cis)-4-amino-5-fluoro-1-[2-(hydroxymethyl)-1.3-oxathiolan-5-yl]-pyrimidin-2(1H)-one, compare WO 92/14743 (=U.S. Pat. Nos. 5,204,466, 5,210,085, 5,539,116, 5,700,937, 5,728,575, 5,814,639, 5,827,727, 5,852,027, 5,892,025, 5,914,331, 5,914,400) and WO 92/18517;

□-L-FDDC=5-(6-amino-2-fluoro-9H-purin-9-yl)-tetrahydro-2-furanmethanol, compare WO 94/27616 (=U.S. Pat. Nos. 5,627,160, 5,561,120, 5,631,239 and 5,830,881);

L-FMAU=1-(2-deoxy-2-fluoro-β-L-arabinofuranosyl)-5-methyl-pyrimidine-2.4(1H,3H)-dione, compare WO 99/05157, WO 99/05158 and U.S. Pat. No. 5,753,789.

The further preferred embodiment of the invention relates to combinations of A) the above isoxazoles (1) and B) (ii) lamivudine.

Other preferred HBV-antiviral agents B comprise, for example, phenylpropenamides of the formula

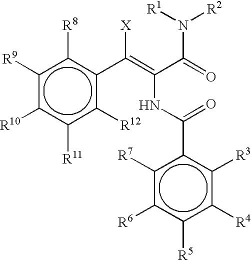

in which

R¹ and R² are, independently of one another, C₁-C₄-alkyl or form, together with the nitrogen atom on which they are located, a ring having 5 to 6 ring atoms which comprise carbon and/or oxygen,

R³-R¹² are, independently of one another, hydrogen, halogen, C₁-C₄-alkyl, optionally substituted C₁-C₄-alkoxy, nitro, cyano or trifluoromethyl,

R¹³ is hydrogen, C₁-C₄-alkyl, C₁-C₇-acyl or aralkyl and

X is halogen or optionally substituted C₁-C₄-alkyl,

and the salts thereof.

These phenylpropenamides and processes for their preparation are disclosed in WO 98/33501, the disclosure of which is incorporated herein by reference. AT-61 is the compound of the above formula in which X is chlorine, A is 1-piperidinyl and each of Y and Z is phenyl.

Preferred immunomodulators C) comprise, for example, all interferons such as α-, β- and γ-interferons, in particular also α-2a- and α-2b-interferons, interleukins such as interleukin-2, polypeptides such as thymosin-α-1 and thymoctonan, imidazo-quinoline derivatives such as ®Levamisole, immunoglobulins and therapeutic vaccines.

Another preferred embodiment of the invention relates to combinations of A) at least one isoxazole, B (i) at least one dihydropyrimidine, (ii) lamivudine, and, where appropriate, C) interferon.

It was not predictable that the combinations of the invention inhibit the replication of the HBV virus considerably better than the agents known from the prior art or the known combinations thereof. The use of the combinations of the invention offers valuable advantages in the treatment of HBV-induced disorders compared with monotherapy with the individual compounds, i.e. mainly a synergistic antiviral activity, but also good tolerability of the combinations of the invention in the range of toxicity at which 50% of the cells survive (“Tox-50”)—compared with the Tox-50 of the individual components.

It has generally proved to be advantageous both in human and in veterinary medicine to administer the compounds (I) and the combinations according to the invention in total amounts of about 0.5 to about 500, preferably 1 to 100, mg/kg of body weight every 24 hours, where appropriate in the form of a plurality of single doses, to achieve the desired results. A single dose contains the active substance or the active substances preferably in amounts of about 1 to about 80, in particular 1 to 30, mg/kg of body weight. However, it may be necessary to deviate from the dosages mentioned, in particular depending on the species and the body weight of the subject to be treated, the nature and severity of the disorder, the type of preparation and mode of administration of the medicament, and the time or interval within which administration takes place.

The ratio of amounts of components A, B and, where appropriate, C in the combinations according to the invention may vary within wide limits; it is preferably 5 to 1000 mg of A/5 to 500 mg of B, in particular 10 to 500 mg of A/20 to 400 mg of B and, in addition, 5 to 1000 mg of A/5 to 500 mg of B and/or 1 to 10 million I.U. (international units) of C.

The component C which is present where appropriate can preferably be used in amounts of, in particular, 2 to 7 million I.U., about three times a week for a period of up to one year.

The compounds (I) and the combinations according to the invention should generally be present in the abovementioned pharmaceutical preparations in a concentration of about 0.1 to 99.5, preferably about 0.5 to 95, % by weight of the complete mixture.

The present invention includes pharmaceutical preparations which, besides non-toxic, inert pharmaceutically suitable carriers, contain one or more compounds (I) or one or more combinations according to the invention or which consist of a compound (I) or combinations according to the invention, and to processes for producing these preparations.

The abovementioned pharmaceutical preparations may, besides the compounds (I) or besides the combinations according to the invention, also contain other pharmaceutical active substances.

The abovementioned pharmaceutical preparations can be produced by known methods, for example by mixing the active substance or active substances with the carrier(s).

The active substances may act systemically and/or locally. For this purpose, they can be administered in a suitable way, such as, for example, by the oral, parenteral, pulmonary, nasal, sublingual, lingual, buccal, rectal, transdermal, conjunctival or otic route or as implant. The active substances can be administered in administration forms suitable for these administration routes.

Suitable for oral administration are administration forms which deliver the active substances rapidly and/or in a modified manner, such as, for example, tablets without or with (for example enteric) coating, capsules, coated tablets, granules, pellets, powders, emulsions, suspensions and solutions.

Parenteral administration can take place with avoidance of an absorption step (intravenous, intraarterial, intracardiac, intraspinal or intralumbar) or with inclusion of an absorption (intramuscular, subcutaneous, intracutaneous, percutaneous, or intraperitoneal). Administration forms suitable for parenteral administration are, inter alia, preparations for injection and infusion in the form of solutions, suspensions, emulsions, lyophilizates and sterile powders.

Suitable for the other routes of administration are, for example, pharmaceutical forms for inhalation (inter alia powder inhalers, nebulizers), nasal drops/solutions, sprays; tablets or capsules for lingual, sublingual or buccal administration, suppositories, preparations for the ears and eyes, vaginal capsules, aqueous suspensions (lotions, shaking mixtures), lipophilic suspensions, ointments, creams, milk, pastes, dusting powders or implants.

The active substances can be converted in a manner known per se into the stated administration forms. This takes place with use of inert non-toxic, pharmaceutically suitable excipients. These include, inter alia, carriers (for example microcrystalline cellulose), solvents (for example liquid polyethylene glycols), emulsifiers (for example sodium dodecyl sulfate), dispersants (for example polyvinylpyrrolidone), synthetic and natural biopolymers (for example albumin), stabilizers (for example antioxidants such as ascorbic acid), colorings (for example inorganic pigments such as iron oxides) or masking flavors and/or odors.

The areas of indication of the compounds (I) and combinations of the invention comprise:

1. the treatment of acute and chronic viral infections which may lead to infectious hepatitis, for example infections with hepatitis B viruses; particular preference is given to the treatment of chronic hepatitis B infections and the treatment of acute hepatitis B viral infection;

2. the treatment of acute and chronic HBV infections where there is coinfection with the hepatitis delta virus; and

3. the treatment of infections associated with organ transplantations, especially with liver transplantations.

The invention therefore further relates to the compounds (I) for controlling diseases.

The invention further relates to medicaments comprising at least one compound (I) and, where appropriate, other pharmaceutical active substances.

The invention further relates to the use of the compounds (I) for producing a medicament for the treatment and prophylaxis of viral diseases, in particular of hepatitis B.

The invention therefore further relates to the combinations defined above for controlling diseases.

The invention further relates to medicaments comprising at least one of the combinations defined above and, where appropriate, other pharmaceutical active substances.

The invention further relates to the use of the combinations defined above for producing medicaments for the treatment and prophylaxis of the diseases described above, preferably of viral diseases, in particular of hepatitis B.

The percentage data in the following examples are based in each case on weight unless indicated otherwise.

### EXAMPLES

### 1. Preparation Examples

### Example 1

### N-(4-Fluoro-3-methylphenyl)-5-isopropyl-3-methylisoxazole-4-carboxamide

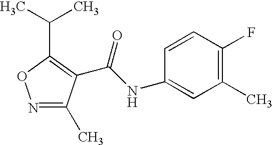

A solution of 10.97 g (69.3 mmol) of ethyl isobutyryl acetate and 4.93 g (69.3 mmol) of pyrrolidine in 50 ml of toluene is heated to reflux in an apparatus with a water trap for 3 hours. The toluene is then removed under reduced pressure, and the residue is dissolved in a mixture of 5.73 g (76.3 mmol) of nitroethane, 28 ml ((201 mmol) of triethylamine and 120 ml of chloroform. This solution is cooled to 5° C., and a solution of 11.7 g (76.3 mmol) of phosphorus oxychloride in 20 ml of chloroform is added dropwise. After the addition is complete, and the mixture is stirred at room temperature for 15 hours and poured into 100 ml of ice-water. The organic phase is separated off, washed successively with 6M hydrochloric acid, 5% strength sodium hydroxide solution, water and saturated aqueous NaCl solution and dried over sodium sulfate. Removal of the solvent by distillation and chromatography on silica gel (mobile phase dichloromethane) afford 7.52 g (55%) of ethyl 5-isopropyl-3-methylisoxazole-4-carboxylate as a colorless oil. ¹H-NMR (300 MHz, DMSO-D₆): δ=1.28 (d, 6H) ppm, 1.31 (t, 3H) ppm, 2.35 (s, 3H) ppm, 3.71 (Quint., 1H) ppm, 4.27 (q, 2H) ppm.

A mixture of 7.5 g (38.0 mmol) of the ester, 70 ml of ethanol, 20 ml of water and 3.04 g (76.1 mmol) of sodium hydroxide is heated to reflux for 2 hours. After cooling, most of the ethanol is removed by distillation under reduced pressure. The aqueous phase is acidified with concentrated hydrochloric acid and then extracted several times with dichloromethane. The combined extracts are dried over sodium sulfate and the solvent is removed. The residue is stirred with petroleum ether. 5.13 g (80%) of 5-isopropyl-3-methylisoxazole-4-carboxylic acid are isolated as a colorless solid by filtering off and drying under reduced pressure. ¹H-NMR (200 MHz, DMSO-D₆): δ=1.25 (d, 6H) ppm, 2.60 (s, 3H) ppm, 3.39 (Quint., 1H) ppm.

MS (DCI/NH₃): 170 [M+H]⁺.

7.03 g (59.1 mmol) of thionyl chloride are added to 2 g (11.8 mmol) of the acid described. The mixture is heated to reflux with stirring until gas evolution ceases (about 1 hour). The thionyl chloride is removed under reduced pressure, and the resulting acid chloride (brown oil) is reacted further without purification.

A mixture of 56.3 mg (0.3 mmol) of the acid chloride, 37.5 mg (0.3 mmol) of 4-fluoro-3-methylanilline and 2.4 ml of 1,2-dichloroethane is mixed with 124 mg of morpholinomethyl-polystyrene (loading 3.69 mmol/g) and stirred at room temperature for 16 hours. The resin is filtered off and washed with dichloromethane. Removal of the volatile constituents under reduced pressure affords 80 mg (96%) of N-(4-fluoro-3-methylphenyl)-5-isopropyl-3-methylisoxazole-4-carboxamide as a colorless solid.

LC-MS (C18 column, 50×2.1 mm, 3.5 μm; gradient acetonitrile +0.1% formic acid [A], water +0.1% formic acid [B]: up to 4 min A/B=1:9, 4-6 min A/B=9:1; flow rate 0.5 ml/min; ionization ESI positive): Rt 4.3 min, m/z 276 [M]⁺.

### Example 2

### N-(4-Fluoro-3-methylphenyl)-3,5-dimethylisoxazole-4-carboxamide

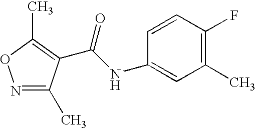

A solution of 2.53 g (18.8 mmol) of 4-fluoro-3-methylaniline and 2.88 ml (20.7 mmol) of triethylamine in 30 ml of dichloromethane is cooled to 0° C. and a solution of 3.0 g (18.8 mmol) of 3,5-dimethylisoxazole carbonyl chloride in 10 ml of dichloromethane is added dropwise. The solution is stirred at 0° C. for 1 hour and then washed successively with 1M hydrochloric acid, saturated aqueous sodium bicarbonate solution and saturated aqueous NaCl solution. The organic phase is dried over sodium sulfate and the volatile constituents are removed under reduced pressure. The remaining residue is chromatographed on silica gel (dichloromethane/ethyl acetate gradient). N-(4-fluoro-3-methylphenyl)-3,5-dimethylisoxazole-4-carboxamide results as a colorless solid (4.0 g, 86%).

¹H-NMR (200 MHz, CDCl₃): δ=2.30 (s, 3H) ppm, 2.51 (s, 3H) ppm, 2.67 (s, 3H) ppm, 6.97 (t, 1H) ppm, 7.23 (m, 1H) ppm, 7.41 (m, 1H) ppm.

MS (DCI/NH₃): 249 (M+H)⁺.

### Example 3

### N-(4-Fluoro-3-methylphenyl)-3,5-dimethylisoxazole-4-thiocarboxamide

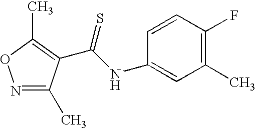

A mixture of 100 mg (0.40 mmol) of N-(4-fluoro-3-methylphenyl)-3,5-dimethylisoxazole-4-carboxamide, 80 mg (0.20 mmol) of Lawesson's reagent and 5 ml of toluene is heated at 90° C. for 1 hour. Removal of the toluene by distillation under reduced pressure is followed by chromatography on silica gel (dichloromethane/ethyl acetate gradient). N-(4-Fluoro-3-methylphenyl)-3,5-dimethylisoxazole-4-thiocarboxamide results as a colorless solid (106 mg, 100%).

¹H-NMR (200 MHz, DMSO-D₆): δ=2.26 (s, 3H) ppm, 2.32 (s, 3H) ppm, 2.52 (s, 3H) ppm, 7.22 (t, 1H) ppm, 7.67 (m, 2H) ppm, 11.65 (s, br, 1H) ppm.

MS (DCI/NH₃): 265 (M+H)⁺.

The compounds of the following examples were synthesized in analogy to examples 1 to 3.

LCMS Methods:

Method A:

C18 column, 150×2.1 mm, 5 μm; gradient acetonitrile+0.1% formic acid [A], water+0.1% formic acid [B]: up to 9 min A/B=1:9, 9-10.1 min A/B=9:1; flow rate 0.5 ml/min; oven temperature 40° C., UV detection 210-350 nm, ionization ESI positive

Method B:

C18 column, 50×2.1 mm, 3.5 μm; gradient acetonitrile+0.1% formic acid [A], water+0.1% formic acid [B]: up to 4 min A/B=1:9, 4-6 min A/B=9:1; flow rate 0.5 ml/min; oven temperature 40° C., UV detection 208-400 nm, ionization ESI positive

Method C:

C18 column, 150×2.1 mm, 5 μm; gradient acetonitrile [A], 0.01N hydrochloric acid [B], water [C]: up to 4 min A/B/C=10:45:45, 4-9 min A/B/C=90:5:5; flow rate 0.6 ml/min; oven temperature 40° C., UV detection 210 nm, ionization ESI positive

| | | Rt | HPLC | 1H-NMR |
|---|---|---|---|---|
| Example | Structure | (min) | method | (200 MHz, DMSO) |
| 4 | 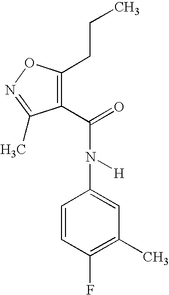 | 7.05 | A | |
| 5 | 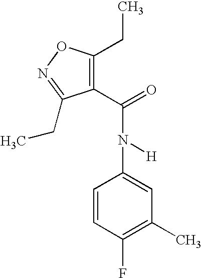 | 3.80 | B | |
| 6 | 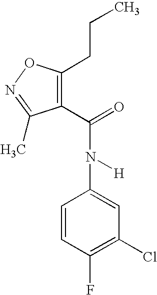 | 7.32 | A | |
| 7 | 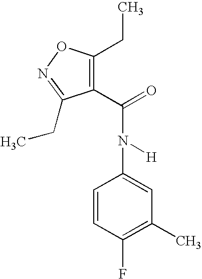 | 3.35 | B | |
| 8 | 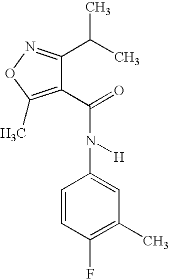 | 4.28 | B | |
| 9 | 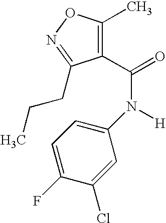 | 4.10 | B | |
| 10 | 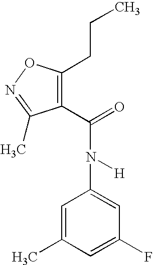 | 7.21 | A | |
| 11 | 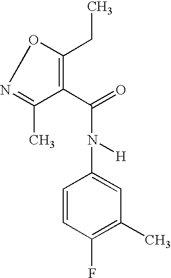 | 4.13 | B | |
| 12 | 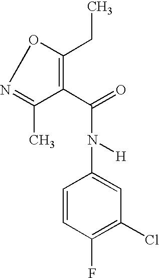 | 4.31 | B | |
| 13 | 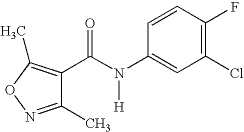 | 5.48 | C | |
| 14 | 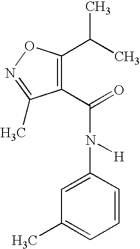 | 4.23 | B | |
| 15 | 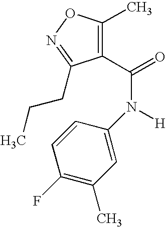 | 3.77 | B | |
| 16 | 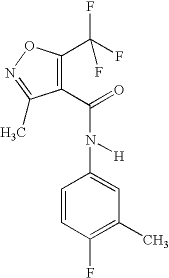 | 4.29 | B | |
| 17 | 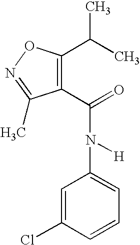 | 4.44 | B | |
| 18 | 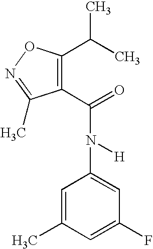 | 4.42 | B | |
| 19 | 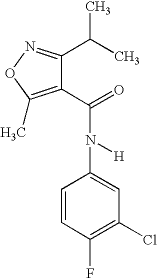 | 4.47 | B | |
| 20 | 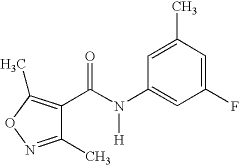 | 5.38 | C | |
| 21 | 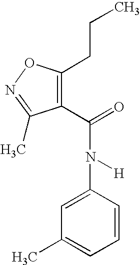 | 6.97 | A | |
| 22 | 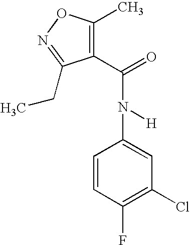 | 3.69 | B | |
| 23 | 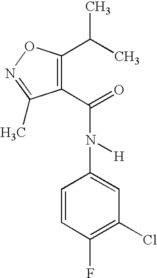 | 4.48 | B | |
| 24 | 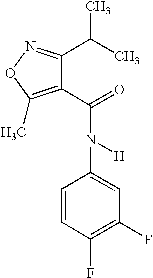 | 4.28 | B | |
| 25 | 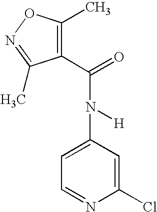 | 4.27 | B | |
| 26 | 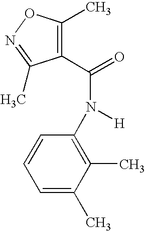 | 4.85 | B | |
| 27 | 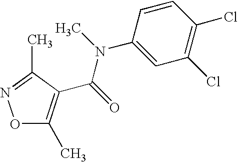 | | | 2.11(s, 3H), 2.14(s, 3H), 3.36(s, 3H), 7.23(dd, 1H), 7.59(d, 1H), 7.73(d, 1H) |
| 28 | 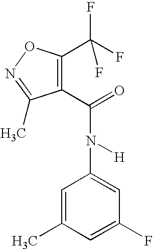 | 4.20 | B | |
| 29 | 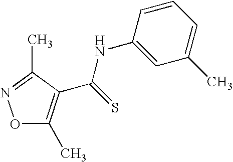 | | | 2.32(s, 3H), 2.33(s, 3H), 2.50(s, 3H), 7.11(d, 1H), 7.33(dd, 1H), 7.63(m, 2H), 11.68(s, br, 1H) |
| 30 | 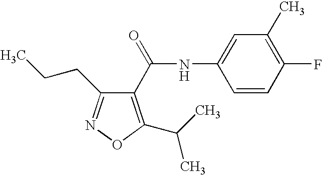 | | | (400MHz, CD₂Cl₂) 1.00(t 3H), 1.32(d, 6H), 1.71-1.83(m, 2H), 2.28(d, 3H), 2.87-2.95(m, 2H), 3.20-3.31(m, 1H), 6.97-7.03(m, 1H), 7.22(broad s, 1H), 7.29-7.36(m, 1H), 7.38-7.42(m, 1H) |
| 31 | 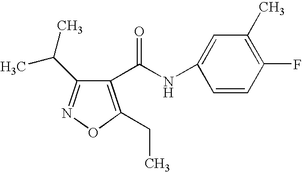 | | | (300MHz, CDCl₃) 1.33-1.41(m, 9H), 2.29(d, 3H), 3.20-3.38(m, 1h), 3.99(q, 2H), 6.98(t, 1H), 7.15(broads, 1H), 7.25-7.30(m, 1H), 7.41(dd, 1H) |

### Example 32

### N-[(3,5-Dimethyl-4-isoxazolyl)methyl]-4-fluoro-3-methylaniline

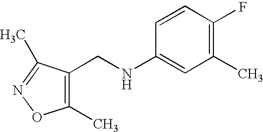

Under argon, 500 mg (2.01 mmol) of the compound from example 2 are dissolved in 30 ml of tetrahydrofuran and, at 0° C., 0.65 g (8.56 mmol, 4.28 ml) of borane/dimethyl sulfide complex is added. The mixture is then heated to boiling for 2 h. 4.13 ml of 1N hydrochloric acid are added and the mixture is stirred under reflux for a further hour. Cooling to room temperature and addition of 12.5 ml of 0.5M sodium hydroxide solution are followed by extraction with ethyl acetate, and the organic phase is washed with saturated sodium chloride solution. It is dried over magnesium sulfate, and the solvent is removed by distillation. The residue is then purified by chromatography on silica gel (1. dichloromethane, 2. cyclohexane:ethyl acetate 6:1) and recrystallized. The target compound is obtained in a yield of 54% (0.253 g).

MS (EI/POS): 234 [M+H]⁺

¹H-NMR (200 MHz, CDCl₃): δ=2.22 (d, 3H) ppm; 2.27 (s, 3H) ppm; 2.38 (s, 3H) ppm, 3.30 (broad s, 1H) ppm; 3.95 (s, 2H) ppm; 6.35-6.50 (m, 2H) ppm; 6.85 (t, 1H) ppm.

The following example 33 is prepared in analogy to the method for example 32 starting from the compound of example 30:

### Example 33

### 4-Fluoro-N-[(5-isopropyl-3-propyl-4-isoxazolyl)methyl]-3-methylaniline

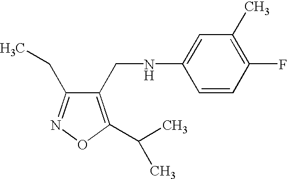

Yield: 13%

MS (DCI/NH₃)=291 [M+H]⁺, 308 [M+NH₄]⁺

¹H-NMR (300 MHz, CDCl₃): δ=0.95 (t, 3H) ppm; 1.33 (d, 6H) ppm; 1.60-1.80 (m, 2H) ppm; 2.21 (d, 3H) ppm; 2.70 (t, 2H) ppm; 2.95-3.11 (m, 1H) ppm; 3.22 (broad s, 1H) ppm; 3.95 (s, 2H) ppm; 6.37-6.50 (m, 2H) ppm; 6.86 (t, 1H) ppm.

### Example 34

### Stage A

### 3,5-Dimethyl-4-isoxazolamine

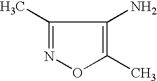

12.00 g (84.44 mmol) of 3,5-dimethyl-4-nitroisoxazole are introduced into 430 ml of water, and 106.15 g (1.984 mol) of ammonium chloride are added. At 4° C., 46.93 g (7.17 mol) of zinc are added over the course of 2 h, ethyl acetate is added to the reaction solution, and the organic phase is filtered through Celite. After drying over magnesium sulfate, the solvent is removed by distillation, and the target compound is obtained in a yield of 86% (8.10 g).

¹H-NMR (300 MHz, CDCl₃): δ=2.20 (s, 3H) ppm; 2.28 (s, 3H) ppm; 2.51 (broad s, 2H) ppm.

### Stage B

### N-(3,5-Dimethyl-4-isoxazolyl)-N′-(4-fluoro-3-methylphenyl)urea

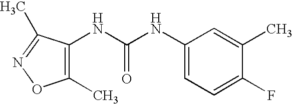

1.25 g (10.00 mmol) of 3-methyl-4-fluoroaniline are dissolved in 40 ml of dichloromethane, and 4.29 g (20.00 mmol) of 1,8-bis(dimethylamino)naphthalene are added. At 0° C., 0.72 ml (6.00 mmol) of trichloromethyl chloroformate in 10 ml of dimethylmethane is added dropwise, and the mixture is stirred at room temperature for 1 h. It is then diluted with 50 ml of dichloromethane and washed with ice-water, 1N hydrochloric acid and saturated sodium bicarbonate solution. After drying over magnesium sulfate, 1.12 g (10.00 mmol) of the amine from stage A are added, and the mixture is heated to boiling for 4 h. The precipitate is filtered off with suction, washed with dichloromethane and recrystallized from ethanol. The target compound is obtained in a yield of 27% (0.71 g).

MS (DCI/NH₃): 264 [M+H]⁺

¹H-NMR (200 MHz, D₆-DMSO): δ=2.10 (s, 3H) ppm; 2.20 (d, 3H) ppm; 2.26 (s, 3H) ppm; 7.00 (t, 1H) ppm; 7.28-7.30 (m, 1H) ppm; 7.34 (dd, 1H) ppm; 7.68 (broad s, 1H) ppm; 8.72 (broad s, 1H) ppm.

### Example 35

### N-(3,5-Dimethyl-4-isoxazolyl)-N′-(4-fluoro-3-methylphenyl)thiourea

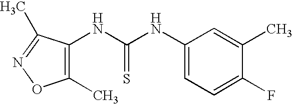

1.25 g (10.00 mmol) of 3-methyl-4-fluoroaniline are dissolved in 50 ml of toluene, and 2.18 g (11.00 mmol) of N,N′-thiocarbonyldiimidazole are added. The mixture is then heated to boiling for 45 minutes. After cooling to 50° C., 1.12 g (10.00 mmol) of the compound from example 34 (stage A) are added, and the reaction solution is stirred at 70° C. for 4 h. The residue after removal of the solvent by distillation is stirred with ethyl acetate, and the crystals are filtered off with suction and recrystallized from ethanol. The target compound is obtained in a yield of 54% (1.50 g).

MS (DCI/NH₃): 280 [M+H]⁺

¹H-NMR (200 MHz, D₆-DMSO): δ=2.10 (s, 3H) ppm; 2.20 (d, 3H) ppm; 2.25 (s, 3H) ppm; 7.05-7.18 (m, 1H) ppm; 7.18-7.38 (m, 2H) ppm; 8.95 (broad s, 1H) ppm; 9.80 (broad s, 1H) ppm.

### Example 36

### 3-(3,5-Dimethyl-4-isoxazolyl)-N-(4-fluoro-3-methylphenyl)propanamide

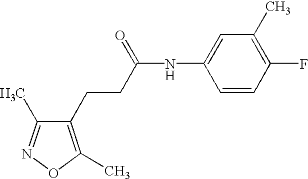

1.00 g (7.99 mmol) of 4-fluoro-3-methylaniline is dissolved in 20 ml of dichloromethane, and 1.21 g (11.99 mmol) of triethylamine are added. At 0° C., a solution of 1.50 g (7.99 mmol) of 3-(3,5-dimethyl-4-isoxazolyl)propanoyl chloride in 10 ml of dichloromethane is added. The mixture is stirred at 0° C. for 1 hour and then washed with water, 1N hydrochloric acid, saturated sodium bicarbonate solution and saturated sodium chloride solution. Drying over magnesium sulfate is followed by filtration through silica gel, and the filtrate is concentrated, and the residue is removed and recrystallized from ethyl acetate/n-pentane. The target compound is obtained in a yield of 54% (1.19 g).

Melting point: 136-137° C.

### Example 37

### N-[3-(3,5-Dimethyl-4-isoxazolyl)propyl]-N-(4-fluoro-3-methylphenyl)amine

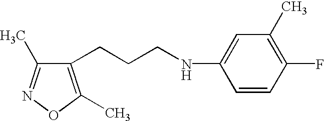

0.50 g (1.81 mmol) of the compound from example 3 is dissolved in 30 ml of tetrahydrofuran and, at 0° C., 0.58 g (7.69 mmol) of borane/dimethyl sulfide complex is added. After stirring under reflux for 2 hours, the mixture is again cooled to 0° C. and, after addition of 3.71 ml of 1N hydrochloric acid, heated to boiling for 1 hour. At room temperature, 11 ml of 1N sodium hydroxide solution are added, and extraction with ethyl acetate is followed by washing with saturated sodium chloride solution. After drying over magnesium sulfate and removal of the solvent by distillation, the target compound is obtained purified by flash chromatography on silica gel (1. cyclohexane, 2. cyclohexane/ethyl acetate 5:1, 2:1) and recrystallization from ethyl acetate/n-pentane in a yield of 58% (0.273 g).

Melting point: 74-76° C.

### 2. Use Examples

The antiviral action of the compounds according to the invention was investigated by methods based on those described by M. A. Sells et al., Proc. Natl. Acad. Sci. 84, 1005-1009 (1987) and B. E. Korba et al., Antiviral Research 19, 55-70 (1992).

The antiviral tests were carried out in 96-well microtitre plates. The first vertical row of the plate received only growth medium and HepG2.2.15 cells. It served as virus control.

Stock solutions of the test compounds (50 mM) were initially dissolved in DMSO, and further dilutions were prepared in the HepG2.2.15 growth medium. The compounds according to the invention were usually pipetted in a test concentration of 100 μM (1st test concentration) in each case into the second vertical test row of the microtitre plate and subsequently diluted in twofold steps 2¹⁰ times in growth medium+2% by weight fetal calf serum (volume 25 μl).

Each well of the microtitre plate then contained 225 μl of HepG2.2.15 cell suspension (5×10⁴ cells/ml) in growth medium+2% by weight of fetal calf serum.

The test mixture was incubated at 37° C. and 5% CO₂ (v/v) for 4 days.

The supernatant was then aspirated off and discarded, and the wells received 225 μl of freshly prepared growth medium. The compounds according to the invention were each added anew as 10-fold concentrated solution in a volume of 25 μl. The mixtures were incubated for a further 4 days.

Before harvesting the supernatants and/or cells to determine the antiviral effect, the HepG2.2.15 cells were examined under the light microscope or by means of biochemical detection methods (for example Alamar Blue stain or Trypan Blue stain) for cytotoxic changes.

The supernatants/cells were then harvested and sucked by means of a vacuum onto 96-well dot-blot chambers covered with a nylon membrane (in accordance with the manufacturer's information).

Cytotoxicity Determination

Substance-induced cytotoxic or cytostatic changes in the HepG2.2.15 cells were detected, for example, under the light microscope as changes in cell morphology. Such substance-induced changes in the HepG2.2.15 cells compared with untreated cells were visible, for example, as cytolysis, vacuolation or altered cell morphology. 50% cytotoxicity (Tox.−50) means that 50% of the cells show a morphology comparable to the corresponding cell control.

The tolerability of some of the compounds according to the invention was additionally tested on other host cells such as, for example, HeLa cells, primary human peripheral blood cells or transformed cell lines such as H-9 cells.

As a rule, the compounds according to the invention were tolerated up to concentrations of 10 μM (Tox. −50).

Determination of the Antiviral Action

After the supernatants or cells had been transferred to the nylon membrane of the blot apparatus (see above), the supernatants of the HepG2.2.15 cells were denatured (1.5 M NaCl/0.5 N NaOH), neutralized (3M NaCl/0.5M Tris HCl, pH 7.5) and washed (2×SSC). The DNA was then baked onto the membrane by incubating the filters at 120° C. for 2-4 hours.

DNA Hybridization

Detection of the viral DNA from the treated HepG2.2.15 cells on the nylon filters was easily carried out with non-radioactive, digoxigenin-labelled hepatitis B-specific DNA probes, each of which was labelled with digoxigenin, purified and employed for the hybridization in accordance with the manufacturer's information.

The prehybridization and hybridization took place in 5×SSC, 1× blocking reagent, 0.1% by weight N-lauroylsarcosine, 0.02% by weight SDS and 100 μg of herring sperm DNA. The prehybridization took place at 60° C. for 30 minutes, and the specific hybridization with 20 to 40 ng/ml of the digoxigenized, denatured HBV-specific DNA took place at 60° C. for 14 hours. The filters were then washed.

Detection of HBV DNA by Digoxigenin Antibodies

The immunological detection of the digoxigenin-labelled DNA took place in accordance with the manufacturer's information:

The filters were washed and prehybridized in a blocking reagent (in accordance with the manufacturer's information). Hybridization was then carried out with an anti-DIG antibody coupled to alkaline phosphatase for 30 minutes. After a washing step, the substrate of alkaline phosphatase, CSPD, was added, incubated with the filters for 5 minutes, then packed in plastic film and incubated at 37° C. for a further 15 minutes.

The chemiluminescence of the hepatitis B-specific DNA signals was visualized by exposing the filters to an X-ray film (incubation depending on signal strength: 10 minutes to 2 hours).

The half-maximum inhibitory concentration (IC-50, 50% inhibitory concentration) was determined as the concentration at which the hepatitis B-specific band was reduced by the compound according to the invention by 50% compared with an untreated sample.

The hepatitis B virus-producing HepG2.2.15 cells with the compounds of the invention surprisingly led to a reduction in viral DNA in the cell culture supernatant which is released by the cells in the form of virions into the cell culture supernatant, or to a reduction in intracellular viral DNA.

Activity data:

| | IC-50 | Tox-50 |
|---|---|---|
| | [μM/l] | [μM/l] |
| Leflunamide | >70 | 70 |
| (comparative) | | |
| Example 2 | 0.2 | >100 |
| Example 12 | 0.5 | >100 |
| Example 16 | 0.75 | >100 |
| Example 21 | 1 | >100 |
| Example 29 | 4 | >100 |

The compounds of the invention show an unpredictable and valuable effect on viruses. They surprisingly have antiviral activity against hepatitis B (HBV) and are thus suitable for the treatment of virus-induced diseases, in particular of acutely and chronically persistent viral infections by HBV. A chronic viral disease caused by HBV may lead to pathological states of varying severity; it is known that chronic hepatitis B viral infection leads in many cases to cirrhosis of the liver and/or hepatocellular carcinoma.

1. A compound of the formula 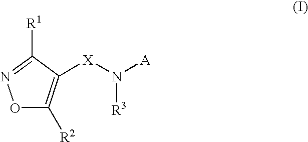
in which
R¹ and R² are, independently of one another, alkyl which is optionally substituted by one or more halogen atoms,
X is a divalent radical from the series consisting of C═Y, —N(R⁴)—C(═Y)—, —CH₂— or a group of the formula —(CH₂)ₙC(═Y)—,
n is an integer from 1 to 4,
R³ and R⁴ are, independently of one another, hydrogen or optionally halogen-substituted alkyl,
Y is an oxygen or sulfur atom and
A is aryl or 6-membered hetaryl which is optionally substituted by 1 to 3 radicals which are selected, independently of one another, from the series halogen, alkyl, alkoxy, alkylthio, alkoxycarbonyl, aminocarbonylamino, mono- and dialkylamino, cyano, amino, mono- and dialkylaminocarbonyl—in the case of substitution in the o position from the series halogen, alkyl, alkoxy, alkylthio.

2. A compound of the formula (I) as claimed in claim 1, in which
R¹ and R² are, independently of one another, optionally halogen-substituted C₁-C₈-alkyl,
X is a divalent radical from the series C═Y and CH₂,
R³ and R⁴ are, independently of one another, hydrogen or optionally halogen-substituted C₁-C₆-alkyl,
Y is an oxygen or sulfur atom and
A is phenyl, pyridyl or pyrimidyl, which are optionally substituted by 1 to 3 radicals from the series halogen, C₁-C₆-alkyl, C₁-C₆-alkoxy, C₁-C₆-alkylthio, C₁-C₆-alkoxycarbonyl, carbamoyl, mono-C₁-C₆-alkylaminocarbonyl, di-C₁-C₆-alkylaminocarbonyl, cyano—in the case of substitution in the o position from the series halogen, C₁-C₆-alkyl, C₁-C₆-alkoxy, C₁-C₆-alkylthio.

3. A compound of the formula (I) as claimed in claim 1, in which
R¹ and R² are, independently of one another, C₁-C₆-alkyl or trifluoromethyl,
X is C═Y,
R³ and R⁴ are, independently of one another, hydrogen or C₁-C₆-alkyl,
Y is an oxygen or sulfur atom and
A is mono- to trisubstituted, phenyl or pyridyl, the substituents of which are selected independently of one another from the series alkyl, halogen, CF₃.

4. A compound of the formula (I) as claimed in claim 1, in which
R¹ and R² are, independently of one another, C₁-C₆-alkyl or trifluoromethyl,
X is C═Y,
R³ and R⁴ are, independently of one another, hydrogen or C₁-C₆-alkyl,
Y is an oxygen or sulfur atom and
A is disubstituted, phenyl or pyridyl, the substituents of which are selected independently of one another from the series alkyl, halogen, CF₃ and phenyl.

5. A compound of the formula (I) as claimed in claim 1, in which
R¹ and R² are, independently of one another, C₁-C₆-alkyl or trifluoromethyl,
X is C═Y,
R³ and R⁴ are, independently of one another, hydrogen or methyl,
Y is an oxygen atom and
A is 3-methyl-4-fluorophenyl or 3-chloro-4-fluorophenyl.

6. A compound selected from the compound of examples 1, 2 and 4 to 9.

7. A process for preparing compounds of the formula (I) as claimed in claims 1 to 6, in which
R¹ to R³ and A have the meanings indicated in claims 1 to 6,
X is C═Y or a group of the formula —(CH₂)ₙC(═Y)—,
Y is an oxygen atom and
n is an integer from 1 to 4,
by reacting acid chlorides of the formula 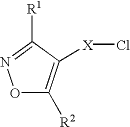
in which
R¹ and R² have the meanings indicated in claims 1 to 6, and
X and Y have the above meanings,
with amines of the formula NHAR³,
in which A and R³ have the meanings indicated above.

8. A process for preparing compounds of the formula (I) in which
R¹ to R³ and A have the meanings indicated in claims 1 to 4, and
X is C═Y and
Y is a sulfur atom,
by treating compounds of the formula (I), in which
R¹ to R³ and A have the meanings indicated above,
X is C═Y and
Y is an oxygen atom,
with Lawesson's reageant.

9. A process for preparing compounds of the formula (I) in which
R¹ to R³ and A have the meanings indicated in claims 1 and 2, and
X is CH₂,
by reducing compounds of the formula (I) in which
R¹ to R³ and A have the meanings indicated above, and
X is C═Y and
Y is an oxygen atom.

10. A process for preparing compounds of the formula (I) in which
R¹ to R⁴ and A have the meanings indicated in claim 1, and
X is —N(R⁴)—C(═Y)— and
Y is an oxygen atom,
by reacting compounds of the formula 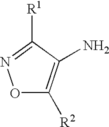
in which
R¹ and R² have the meanings indicated above,
with carbonyl group donors and amines of the formula NHAR³
in which A and R³ have the meanings indicated above.

11. A process for preparing compounds of the formula (I) in which
R¹ to R⁴ and A have the meanings indicated in claim 1, and
X is —N(R⁴)—C(═Y)— and
Y is a sulfur atom,
by reacting compounds of the formula 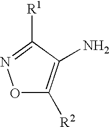
in which
R¹ and R² have the meanings indicated above,
with thiocarbonyl group donors and amines of the formula NHAR³
where A and R³ have the meanings indicated above.

12. A combination of
A) at least one isoxazole,
B) at least one HBV-antiviral active substance different from A and, where appropriate,
C) at least one immunomodulator.

13. A combination as claimed in claim 12, wherein the isoxazole A corresponds to the formula 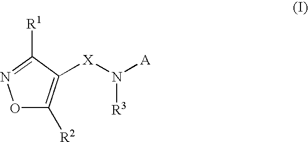
in which
R¹ and R² are, independently of one another, alkyl which is optionally substituted by one or more halogen atoms,
X is a divalent radical from the series C═Y, —N(R⁴)—C(═Y)—, CH₂,
R³ and R⁴ are, independently of one another, hydrogen or alkyl,
Y is an oxygen or sulfur atom and
A is aryl or hetaryl which are optionally substituted by 1 to 3 radicals selected, independently of one another, from the series halogen, alkyl, alkoxy, alkylthio, alkoxycarbonyl, aminocarbonylamino, mono- and dialkylamino, cyano, amino, mono- and dialkylaminocarbonyl.

14. A combination as claimed in claims 12 and 13, wherein the component B comprises at least one dihydropyrimidine of the formula 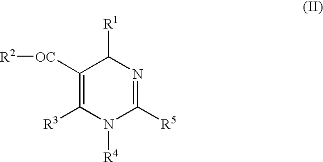
and the isomeric form thereof 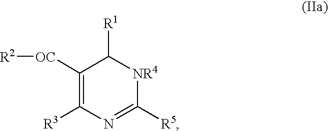
and/or the salts thereof, in which
R¹ is phenyl, furyl, thienyl, triazolyl, pyridyl, cycloalkyl having 3 to 6 carbon atoms or radicals of the formulae 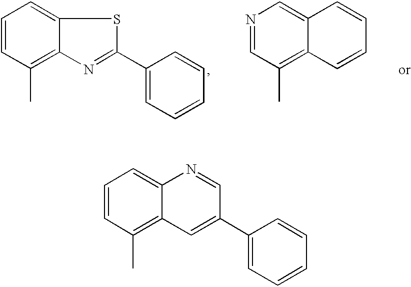
where the ring systems mentioned above are optionally substituted one or more times, identically or differently, by substituents selected from the group of halogen, trifluoromethyl, nitro, cyano, trifluoromethoxy, carboxyl, hydroxyl, C₁-C₆-alkoxy, C₁-C₆-alkoxycarbonyl and C₁-C₆-alkyl, where the alkyl radical in turn may be substituted by aryl having 6 to 10 carbon atoms or halogen, and the mentioned ring systems are optionally substituted by —S—R⁶, —NR⁷R⁸, —CO—NR⁹R¹⁰, —SO₂—CF₃ and —A—CH₂—R¹¹,
in which
R⁶ is optionally halogen-substituted phenyl,
R⁷ to R¹⁰ are, independently of one another, hydrogen, phenyl, hydroxy-substituted phenyl, hydroxyl, C₁-C₆-acyl or C₁-C₆-alkyl, where the alkyl radical in turn may be substituted by hydroxyl, C₁-C₆-alkoxycarbonyl, phenyl or hydroxy-substituted phenyl,
A is a radical —O—, —S—, —SO— or —SO₂—,
R¹¹ is phenyl which is optionally substituted one or more times, identically or differently, by substituents selected from the group of halogen, nitro, trifluoromethyl, C₁-C₆-alkyl and C₁-C₆-alkoxy,
R² is a radical of the formulae —XR¹² or —NR¹³R¹⁴,
in which
X is a single bond or oxygen,
R¹² is hydrogen, straight-chain or branched C₁-C₆-alkoxycarbonyl, a straight-chain, branched or cyclic, saturated or unsaturated C₁-C₈-hydrocarbon radical which optionally contains one or two identical or different hetero chain members from the group of —O—, —CO—, —NH—, —N—(C₁-C₄-alkyl)-, —S— or —SO₂— and which is optionally substituted by halogen, nitro, cyano, hydroxyl, aryl having 6 to 10 carbon atoms, aralkyl having 6 to 10 carbon atoms, heteroaryl or a group of the formula —NR¹⁵R¹⁶,
in which R¹⁵ and R¹⁶ are, independently of one another hydrogen, benzyl or C₁-C₆-alkyl,
R¹³ and R¹⁴ are, independently of one another, hydrogen, C₁-C₆-alkyl or cycloalkyl having 3 to 6 carbon atoms,
R³ is hydrogen, amino or a radical of the formula 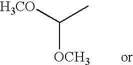
formyl, cyano, hydroxy-substituted C₁-C₆-alkylthio, trifluoromethyl or pyridyl or a straight-chain, branched or cyclic, saturated or unsaturated hydrocarbon radical having up to 8 carbon atoms which is optionally substituted one or more times, identically or differently, by aryloxy having 6 to 10 carbon atoms, azido, halogen, cyano, hydroxyl, carboxyl, C₁-C₆-alkoxycarbonyl, a 5- to 7-membered heterocyclic ring, C₁-C₆-alkylthio or C₁-C₆-alkoxy (where the alkylthio or alkoxy radical may in turn be substituted by azido, amino, hydroxyl) and/or by the group —(CO)ₐ—NR¹⁷R¹⁸,
in which
a is zero or 1,
R¹⁷ and R¹⁸ are, independently of one another, hydrogen or aryl having 6 to 10 carbon atoms, aralkyl having 6 to 10 carbon atoms or C₁-C₆-alkyl, each of which is optionally substituted by C₁-C₆-alkoxycarbonyl, amino, hydroxyl, phenyl or benzyl, where phenyl and benzyl are optionally substituted one or more times, identically or differently, by hydroxyl, carboxyl, C₁-C₆-alkyl or C₁-C₆-alkoxy, and/or C₁-C₆-alkyl is optionally substituted by —NH—CO—CH₃ or —NH—CO—CF₃,
or
R¹⁷ and R¹⁸ together with the nitrogen atom on which they are located are a morpholinyl, piperidinyl or pyrrolidinyl ring,
or
R³ is optionally methoxy-substituted phenyl
or
R² and R³ together are a radical of the formula 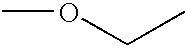
R⁴ is hydrogen, C₁-C₄-alkyl, C₂-C₄-alkenyl, benzoyl or acyl having 2 to 6 carbon atoms, preferably hydrogen, methyl, benzoyl or C₂-C₆-acyl, and
R⁵ is pyridyl, pyrimidyl or pyrazinyl, each of which may be substituted up to 3 times, identically or differently, by halogen, hydroxyl, cyano, trifluoromethyl, C₁-C₆-alkoxy, C₁-C₆-alkyl, C₁-C₆-alkylthio, carbalkoxy, C₁-C₆-acyloxy, amino, nitro, mono- or di-C₁-C₆-alkylamino.

15. A combination as claimed in claim 14, wherein the component B comprises at least one compound of the formulae 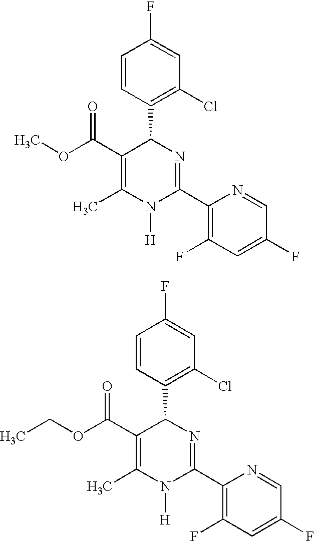
their isomeric forms and/or their salts.

16. A combination as claimed in claim 14, wherein the component B comprises at least one compound of the formula 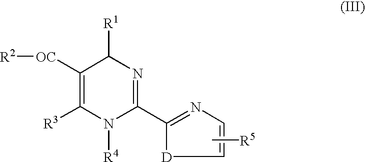
and the isomeric form thereof 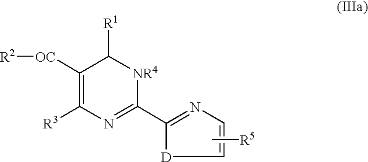
and/or the salts thereof, in which
R¹ is phenyl, furyl, thienyl, pyridyl, cycloalkyl having 3 to 6 carbon atoms or a radical of the formulae 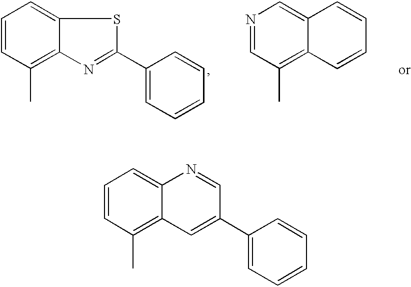
where the ring systems mentioned above are optionally substituted one or more times, identically or differently, by substituents selected from the group of halogen, trifluoromethyl, nitro, cyano, trifluoromethoxy, carboxyl, hydroxyl, C₁-C₆-alkoxy, C₁-C₆-alkoxycarbonyl and C₁-C₆-alkyl, where the alkyl radical in turn may be substituted by aryl having 6 to 10 carbon atoms or halogen, and/or the mentioned ring systems are optionally substituted by groups of the formulae —S—R⁶, —NR⁷R⁸, —CO—NR⁹R¹⁰, —SO₂—CF₃ and —A—CH₂—R¹¹,
in which
R⁶ is optionally halogen-substituted phenyl,
R⁷ to R¹⁰ are, independently of one another, hydrogen, phenyl, hydroxy-substituted phenyl, hydroxyl, C₁-C₆-acyl or C₁-C₆-alkyl, where the alkyl radical in turn may be substituted by hydroxyl, C₁-C₆-alkoxycarbonyl, phenyl or hydroxy-substituted phenyl,
A is a radical —O—, —S—, —SO— or —SO₂—,
R¹¹ is phenyl which is optionally substituted one or more times, identically or differently, by substituents selected from the group of halogen, nitro, trifluoromethyl, C₁-C₆-alkyl and C₁-C₆-alkoxy,
R² is a radical of the formulae —OR¹² or —NR¹³R¹⁴,
in which
R¹² is hydrogen, C₁-C₆-alkoxycarbonyl or a straight-chain, branched or cyclic, saturated or unsaturated C₁-C₈-hydrocarbon radical which optionally contains one or two identical or different hetero chain members from the group of —O—, —CO—, —NH—, —N—(C₁-C₄-alkyl)-, —S— and —SO₂— and which is optionally substituted by halogen, nitro, cyano, hydroxyl, aryl having 6 to 10 carbon atoms or aralkyl having 6 to 10 carbon atoms, heteroaryl or a group of the formula —NR¹⁵R¹⁶,
in which
R¹⁵ and R¹⁶ are, independently of one another, hydrogen, benzyl or C₁-C₆-alkyl,
R¹³ and R¹⁴ are, independently of one another, hydrogen, C₁-C₆-alkyl or cycloalkyl having 3 to 6 carbon atoms,
R³ is hydrogen, amino or a radical of the formula 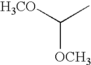
or formyl, cyano, hydroxy-substituted C₁-C₄-alkylthio, trifluoromethyl or a straight-chain, branched or cyclic, saturated or unsaturated hydrocarbon radical having up to 8 carbon atoms, which is optionally substituted one or more times, identically or differently, by aryloxy having 6 to 10 carbon atoms, azido, cyano, hydroxyl, carboxyl, C₁-C₆-alkoxycarbonyl, a 5- to 7-membered heterocyclic ring, C₁-C₆-alkylthio or C₁-C₆-alkoxy (where the alkylthio or alkoxy radical in turn can be substituted by azido, amino or hydroxyl) and/or by the group —(CO)ₐ—NR¹⁷R¹⁸,
in which
a is zero or 1,
R¹⁷ and R¹⁸ are, independently of one another, hydrogen or aryl, aralkyl having 6 to 10 carbon atoms or C₁-C₆-alkyl, which are optionally substituted by C₁-C₆-alkoxycarbonyl, amino, hydroxyl, phenyl or benzyl, where phenyl and benzyl are optionally substituted one or more times, identically or differently, by hydroxyl, carboxyl, C₁-C₆-alkyl or C₁-C₆-alkoxy, and/or C₁-C₆-alkyl is optionally substituted by —NH—CO—CH₃ or —NH—CO—CF₃,
or
R¹⁷ and R¹⁸ together with the nitrogen atom on which they are located are a morpholinyl, piperidinyl or pyrrolidinyl ring,
D is an oyxgen or sulfur atom and
R⁵ is hydrogen, halogen or straight-chain or branched alkyl having up to 6 carbon atoms.

17. A combination as claimed in claims 12 to 16, wherein the component B comprises at least one HBV polymerase inhibitor.

18. A combination as claimed in claims 12 to 16, wherein the component B comprises lamivudine.

19. A combination as claimed in claims 12 to 16, wherein the component B comprises at least one compound of the formula 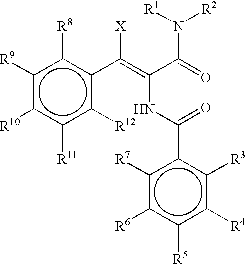
and/or the salts thereof, in which
R¹ and R² are, independently of one another, C₁-C₄-alkyl or form, together with the nitrogen atom on which they are located, a ring having 5 to 6 ring atoms which comprise carbon and/or oxygen,
R³-R¹² are, independently of one another, hydrogen, halogen, C₁-C₄-alkyl, optionally substituted C₁-C₄-alkoxy, nitro, cyano or trifluoromethyl,
R¹³ is hydrogen, C₁-C₄-alkyl, C₁-C₇-acyl or aralkyl and
X is halogen or optionally substituted C₁-C₄-alkyl.

20. A combination as claimed in claims 12 to 19, wherein the component B (i) comprises a dihydropyrimidine and/or (ii) an HBV polymerase inhibitor.

21. A combination as claimed in claims 12 to 19, wherein the component B comprises at least one compound of the formula 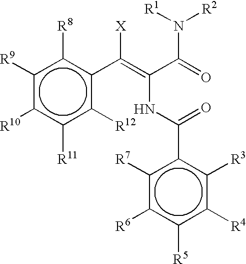
and/or the salts thereof, in which
R¹ and R² are, independently of one another, C₁-C₄-alkyl or form, together with the nitrogen atom on which they are located, a ring having 5 to 6 ring atoms which comprise carbon and/or oxygen,
R³-R¹² are, independently of one another, hydrogen, halogen, C₁-C₄-alkyl, optionally substituted C₁-C₄-alkoxy, nitro, cyano or trifluoromethyl,
R¹³ is hydrogen, C₁-C₄-alkyl, C₁-C₇-acyl or aralkyl and
X is halogen or optionally substituted C₁-C₄-alkyl.

22. A combination as claimed in claim 21,
in which
X is chlorine, A is 1-piperidinyl and each of Y and Z is phenyl.

23. A combination as claimed in claims 12 to 22, wherein the immunomodulator C comprises interferons.

24. A combination of A) at least one oxazole, B) (i) at least one dihydropyrimidine, (ii) lamivudine and, where appropriate, C) at least one interferon.

25. A process for producing the combinations as claimed in claims 12 to 24, characterized in that the components A, B and, where appropriate, C are combined or prepared in a suitable way.

26. A compound as claimed in claims 1 to 6 and a combination as claimed in claims 12 to 24 for controlling diseases.

27. A medicament comprising at least one compound as claimed in claims 1 to 6 or at least one combination as claimed in claims 12 to 24 and, where appropriate, other pharmaceutical active substances.

28. The use of compounds of claims 1 to 6 or of combinations as claimed in claims 12 to 24 for producing a medicament for the treatment and prophylaxis of viral diseases.

29. The use as claimed in claim 28 for producing a medicament for the treatment and prophylaxis of hepatitis B.
